(19) United States
(12) Patent Application Publication
(10) Pub. No.: US 2005/0038607 A1
(43) Pub. Date: Feb. 17, 2005

(54) Method for identification pharmacophores

(76) Inventor: Andreas Schuppert, Kurten (DE)

Correspondence Address: Norris McLaughlin & Marcus, 220 East 42nd Street, 30th Floor, New York, NY, 10017, US

(21) Appl. No.: 10/494,845

(22) PCT Filed: Nov. 11, 2002
(86) PCT No.: PCT/EP02/12549

(30) Foreign Application Priority Data
Nov. 15, 2001 (DE) 101 56 245.4

(51) Int. Cl. G06F 19/00; G01N 33/48; G01N 33/50; G01N 31/00

(52) U.S. Cl. 702/19; 702/22

(57) ABSTRACT

The present invention relates to a method for identifying a molecular pharmacophore, generally comprising the following steps: inputting descriptors of chemical compounds, and assigning effects or results (Rp) of each descriptor. Each descriptor comprises a number of variables (V₁, V₂, . . . , Vₙ). Both binary and ternary variations of the variables are determined, and the binary variations are assigned to an active entity of a putative pharmacophore. Variable pair candidates from the ternary variations are determined for assignment to a common active entity, the common active entity having two or more variables, and further determining a set of variables for each variable pair candidate which contains such variables, which, when the variable pair candidate is assigned to the common active entity, have to be assigned to an active entity other than the common active entity. Conflict-free clusters of sets of variables are used to identify one or more common active entities.

The present invention relates to a method for identifying a molecular pharmacophore, and to a corresponding computer program and computer system.

Searching for molecular pharmacophores from experimental data is a decisive step in searching for new active substances. From the prior art it is known per se to acquire experimental data by examining reactions of a large number of defined substances from a substance library with a previously defined target molecule, referred to as the target. The substances of the substance library are classified in accordance with the reaction with the target. One possible way of classifying them is a binary classification, that is to say for example in accordance with logic “0”, that is to say no reaction, and logic “1”, that is to say a reaction occurs.

In order to develop an active substance, it is decisive to identify pharmacologically relevant subunits (pharmacophores) from the classification of the individual substances and their known chemical structure. This includes also identifying what are referred to as lead structures which are chemically well-defined, coherent subunits of a molecule. A molecular subunit which is relevant for the reaction capability with the target is referred to as a pharmacophore, and in particular as a lead structure. It is irrelevant here whether the contribution of a subunit promotes or inhibits the reaction. The pharmacophores do not necessarily need to form a compact molecular subunit. It is perfectly possible for spatially separated molecular subunits to contribute cooperatively to the effect.

The biological or chemical descriptors or molecular structures are encoded in an input vector. The effect profile is an a priori unknown function which depends on the molecular structure. For this reason, this function is referred to below as structure/effect relationship (SER). The pharmacophore can be derived from its functional of form by linking the effect contributions of the input variables to a small number of effect entities which jointly produce the SER. (cf. J. Bajorath, “Selected Concepts and Investigations in Compound Classification, Molecular Descriptor Analysis, and Virtual Screening”, J. Chem. In. Comput. Sci., 2001, 41, 233-2459.

If a pharmacophore is identified, the active substance can then be optimized by systematic variation thereof. Established methods exist for systematically optimizing an identified pharmacophore.

A combination of different methods are used to identify pharmacophores:
1.) Definition of structural subgroups of the molecular structures (fingerprints) and determination of chemical and/or biological descriptors of the individual molecular structures. Descriptors are molecule-specific chemical variables (for example acidity, number of OH groups etc.) or biological variables (such as toxicity). The fingerprints are coded in the form of binary strings. Here, each position on the string designates a molecular subgroup. A 1 is set at each position on the string if the corresponding subgroup is present in the molecular structure, and otherwise a 0 is set. It has been found empirically that the selection of molecular subgroups is important for the success of identifying the pharmacophore and is the subject matter of current research (cf. U.S. Pat. No. 6,240,374 and U.S. Pat. No. 6,208,942). With respect to the fingerprints, it is possible to encode not only the presence of subgroups but also their relationship in the chemical structure of the molecule. However, the development of optimum generically usable fingerprints is equivalent to identifying pharmacophores and has not yet been achieved.
2.) Data reduction methods are applied to the fingerprints. The most customary ones for this are principal component analysis (PCA) and cluster methods. As a result, the very long strings are considerably reduced, the complexity of the problem of identifying pharmacophores being reduced. As all the methods which exist for this purpose are heuristic and do not contain any information on the effect structure, there is the risk that information which is relevant for the effect will be eliminated during the reduction. Methods for avoiding this systematically do not exist.
3.) Established methods of data mining are applied to the (reduced) data records in order to find structure/effect relationships between fingerprints/descriptors and the pharmacological effect.
   The most customary methods are
      decision trees,
      association rules,
      neural networks.

In the case of decision trees and association rules, combinatorial methods are employed to attempt to arrive at a description of the structure/effect relationship using as few variables as possible. Such a method can therefore be used to separate from one another structural variables which are relevant to the effect and those which are not relevant to the effect. It is a disadvantage that in this context in principle only effect entities which make a positive or negative contribution to the effect irrespective of the allocation of the other structural variables can be identified as relevant. In the frequent case in which an interaction occurs between a plurality of effect entities, it is then possible to identify it only if the overall effect is always promoted or weakened.

In all cases in which a complex interaction occurs between effect entities for structural chemical reasons, said interaction cannot be identified from the methods mentioned above. In these cases, the groupings of structural variables to form effect entities are also not detected. A further disadvantage of the methods is that it is fundamentally possible to detect complex, multi-stage interactions between effect entities.

In contrast to decision trees and association rules, neural networks learn the SER “by heart” by reference to the data present. They are also capable of mapping complex interactions of a large number of variables correctly. Their decisive disadvantage is that they can only supply a formal SER. Explicit information on functional structuring of the SER cannot be acquired. As a result, their contribution to identifying pharmacophores is restricted to permitting a compact representation of the SER as well as interpolations between measured variable allocations. Neural networks cannot make a direct contribution, because of their design, to structuring the SER. A chemically relevant identification of a pharmacophore is therefore possible only to a very limited degree. A second disadvantage is that the high degree of flexibility of neural networks leads to a situation in which, with the highly dimensional data records which are present, the reliability of the prediction by means of a neural network decreases greatly due to overfitting.

Methods which permit the explicit integration of prior knowledge and additionally generate information on the functional structure of the SER from the data are not known.

On the other hand, it has been possible recently to demonstrate the explicit integration of prior knowledge into neural network structures in the form of structured hybrid models and to prove the increase in efficiency in the modeling of complex relationships acquired as a result (cf. A. Schuppert, Extrapolability of Structured Hybrid Models: a Key to Optimization of Complex Processes, in: Proceedings of EquaDiff 99, Fiedler, Groger, Sprekels Eds., World Scientific Publishing, 2000).

Structured hybrid models contain neural networks which are connected to one another in accordance with the functional structure of the SER which is predefined a priori. The effect entities which are implemented as neural networks are then trained in a similar way to unstructured neural networks by reference to the data present. It was possible to show that as a result the problem of overfitting can be greatly reduced. In addition, structured hybrid models permit extrapolation of the data, which is impossible in principle with pure neural networks.

Structured hybrid modeling cannot be applied for the application in pharmacophore identification as long as the functional structure of the SER which is being sought is not known a priori. As this is generally not the case, a corresponding precondition for the use of structured hybrid models is not met. In contrast, clarification of the functional structure of the SER is even the decisive component in searching for pharmacophores.

However, until now it has not been possible to perform a reverse determination of the functional structure of the SER from the available data. In the prior art there is therefore a lack of reliable methods for identifying pharmacophores for a given target.

The invention is therefore based on the object of providing a method for identifying molecular pharmacophores as well as a corresponding computer program and computer system.

The object on which the invention is based is respectively achieved with the features of the independent patent claims. Preferred embodiments of the invention are given in the dependent patent claims.

An advantageous field of application of the present invention is the identification of molecular pharmacophores for the purposes of pharmacological effect analysis. In particular, the invention permits the development of a pharmacological active substance to be speeded up significantly, greatly reducing costs at the same time.

A particular advantage of the invention is that it permits the direct identification of the functional structure of the SER from measured structure/effect data.

According to one preferred embodiment of the invention, it is presumed that the data can be classified in such a way that the effect of each data record is accessible to binary representation, that is to say for the states “not active” and “active”.

According to a further preferred embodiment of the invention, it is also presumed that each effect entity of the pharmacophore can likewise assume only two states, namely “effect” and “inactive”. An effect entity is considered here as a “black box”.

According to a further preferred embodiment of the invention, the effects are divided into more than two classes and coded. In comparison with binary coding, this embodiment permits not only the distinction between “not active” and “active”, but also allows different gradations of the activeness to be included in the evaluation. Correspondingly, it is also possible to permit more than two states for each effect entity.

The invention is based on the recognition that it is a property of structured hybrid models that a precisely defined system of nonvariant sets in the data is associated with each functional structure of the SER. The method according to the invention is based on the fact that the (possibly present) nonvariant sets are filtered out of the data in order to reconstruct the SER from them. (Structured hybrid models are known per se from A. Schuppert, Extrapolability of Structured Hybrid Models: a Key to Optimization of Complex Processes, in: Proceedings of EquaDiff 99, Fiedler, Groger, Sprekels Eds., World Scientific Publishing, 2000.)

In the event of an effect entity being able to assume only two states, namely “active” and “inactive”, there must therefore be clustering of the allocations of the input variables of each effect entity so that under all circumstances the output of the effect entity is logic “0” for all allocations of one of the relevant variables, and always “1” for all the allocations of the other variables. This forced clustering of the allocations of the input variables leads directly to the existence of nonvariant sets in the SER.

A particular advantage of the invention is that the functional structure of the SER can be reconstructed from a predefined system of nonvariant sets of the SER, in particular if the SER has a tree structure. The method according to the invention requires, to calculate the functional structure of the SER, neither the explicit calculation of the precise allocation of the input and output relationships of the individual effect entities nor a combinatorial variation of all the possible functional structures. Owing to this, the method according to the invention is particularly efficient and permits even complex problems to be solved with relatively low calculation complexity.

Preferred exemplary embodiments of the invention are explained in more detail below with reference to the drawings, in which:

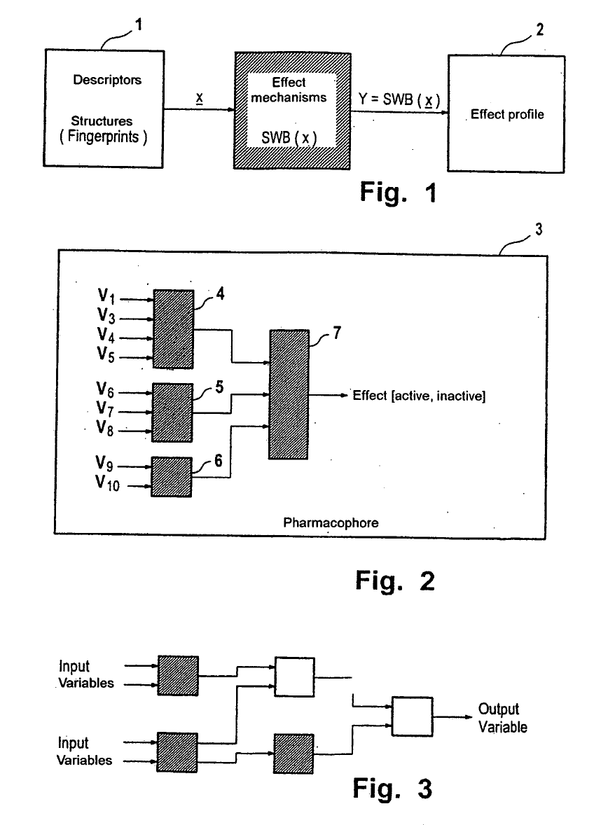

FIG. 1 is a basic illustration of the identification of a pharmacological structure/effect relationship,

FIG. 2 is an example of the formal structure of a pharmacophore,

FIG. 3 is an example of a structured hybrid model,

FIG. 4 is an example of a structure/effect relationship composed of effect entities, each with binary input/output behavior,

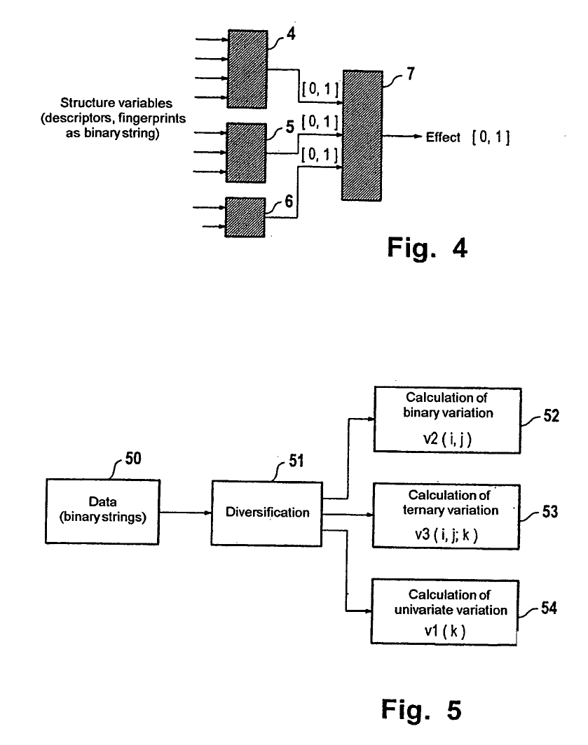

FIG. 5 is a flowchart showing the calculation of different variations of descriptors,

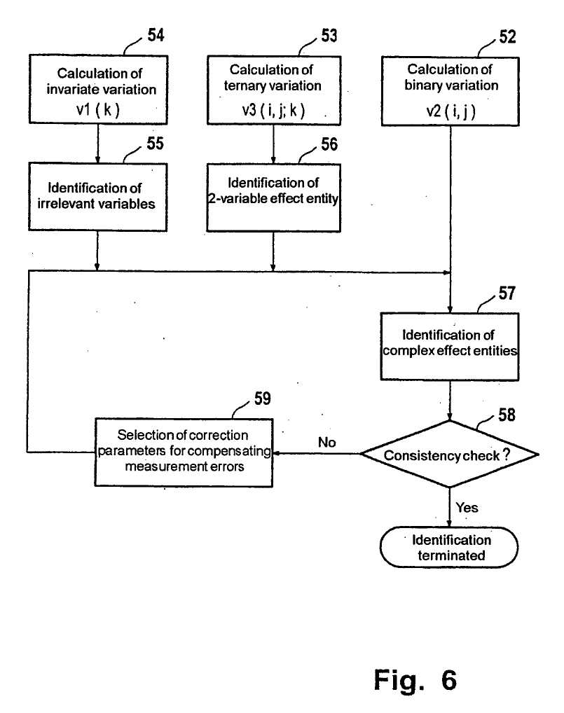

FIG. 6 is a flowchart showing the identification of effect entities,

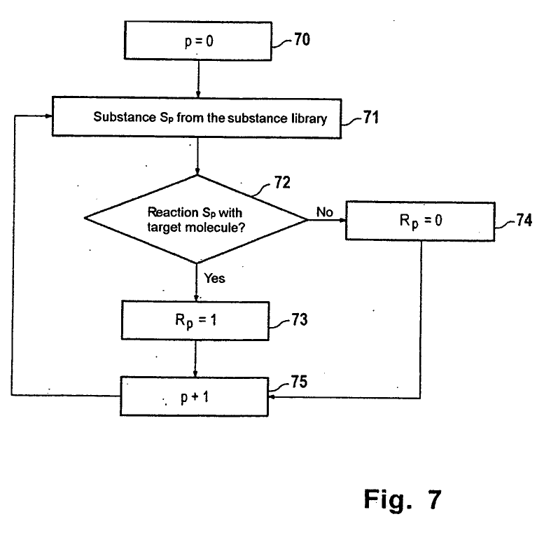

FIG. 7 is a flowchart of a method for experimentally determining substances of a substance library on a target molecule,

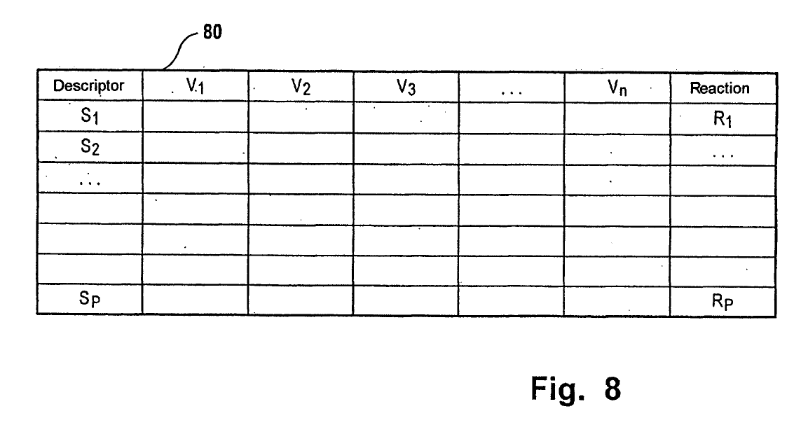

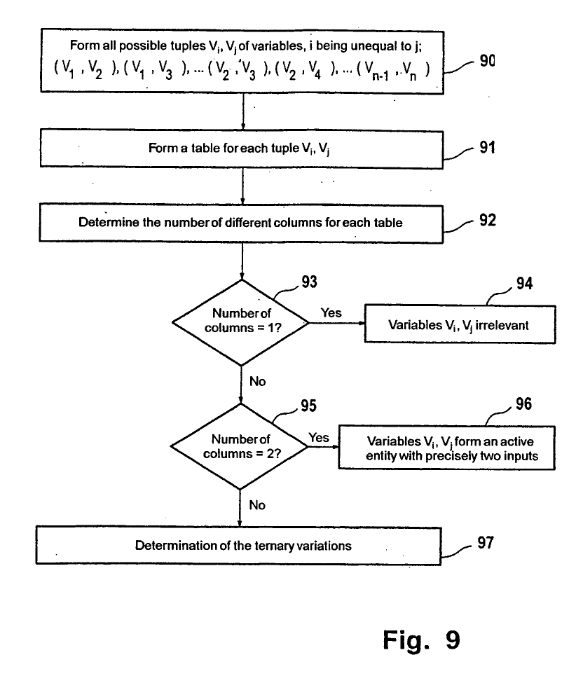

FIG. 8 is a table with descriptors of the substances of the substance library and the experimentally determined reactions,

FIG. 9 is a flowchart of an embodiment of the determination of the binary variations,

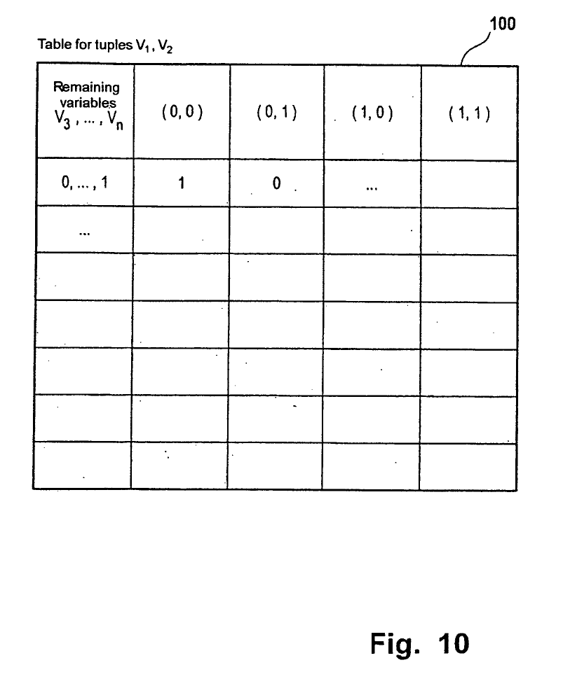

FIG. 10 is a table showing the determination of the binary variations according to FIG. 9,

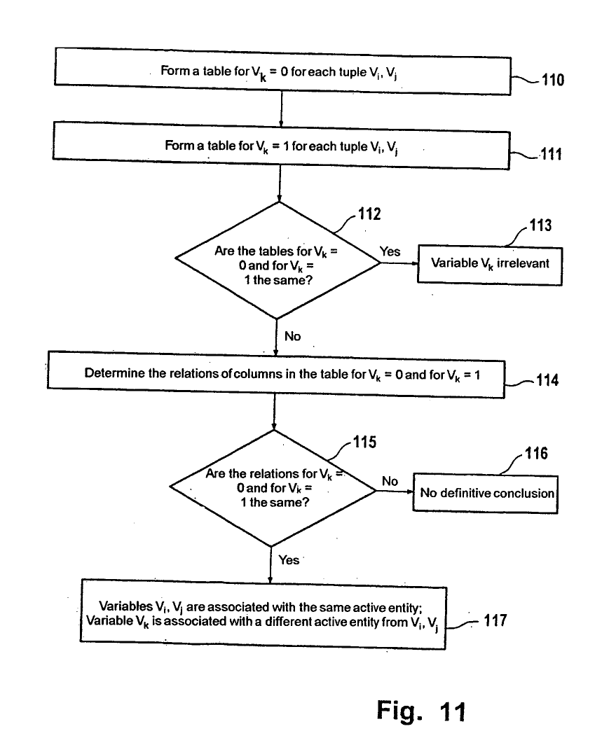

FIG. 11 is a flowchart showing the determination of ternary variations,

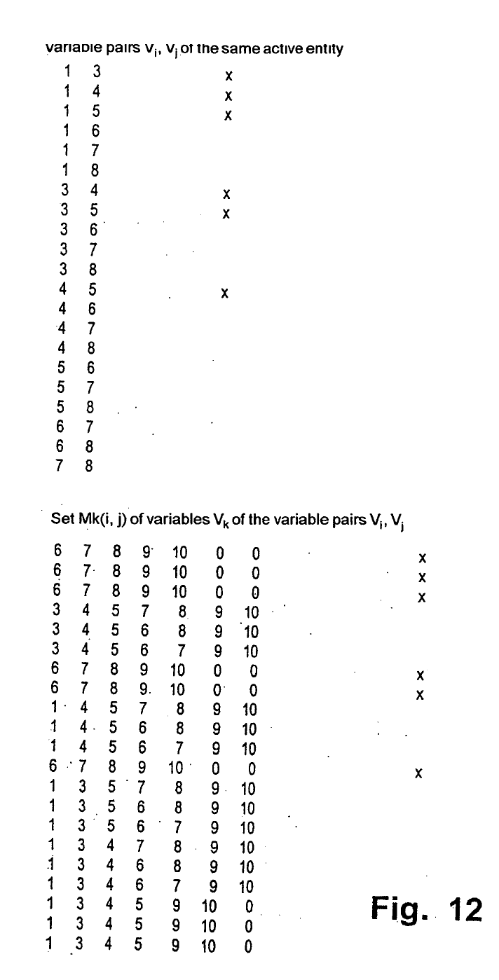

FIG. 12 is a further example of a structure/effect relationship,

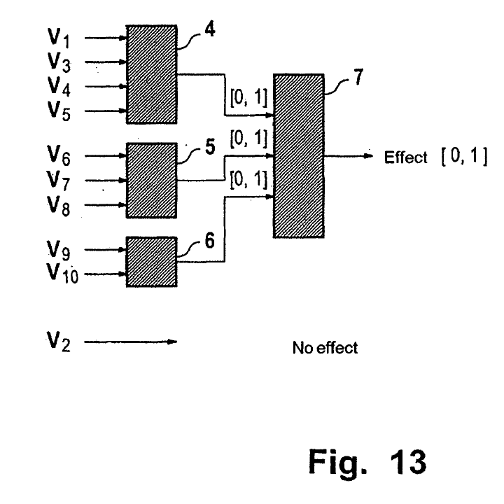

FIG. 13 is a table with variable pair candidates for the assignment to a common active entity and a table of sets of variables for the variable pair candidates with conflict-free clusters.

FIG. 1 illustrates the identification problem on which the invention is based, in particular for pharmacological applications. A database 1 contains the descriptors of the substances of a substance library. The descriptors are preferably binary coded here and describe the structures of the substances. Such descriptors are also referred to as fingerprints. Such fingerprints are known per se from the prior art (cf. J. Bajorath, Selected Concepts and Investigations in Compound Classification, Molecular Descriptor Analysis, and Virtual Screening, J. Chem. In. Comput. Sci., 2001, 41, 233-245).

The descriptors of database 1 are available as vectors x at the output of the database 1 and are mapped onto an effect profile by means of the effect mechanism—to be determined—of the structure/effect relationship SER(x). The effect profile comprises experimentally determined data which is stored in a database 2. In order to determine the effect profile, an experiment is used to determine as far as possible for each individual descriptor whether or not the respective substance reacts with the target molecule, referred to as the target.

The target molecule is therefore used to perform a mapping Y=SER(x) of substances which are described by means of the descriptors onto an effect profile. The identification problem is then to draw inferences about the structure of the SER from the input and output variables of the SER, that is to say from the descriptors and the effect profile.

An SER can be represented as what is referred to as a pharmacophore according to FIG. 2. A pharmacophore may comprise one or more lead structures.

FIG. 2 shows a pharmacophore 3 having the effect entities 4, 5, 6 and 7. The effect entity 4 has, as inputs, the variables V₁, V₃, V₄ and V₅. The effect entity 5 has, as inputs, the variables V₆, V₇ and V₈. The effect entity 6 has the inputs V₉ and V₁₀. The effect entities 4, 5 and 6 each have an output which is linked to an input of the effect entity 7. The output of the effect entity 7 then indicates the overall effect, that is to say, “active” or “inactive”.

FIG. 3 shows an example of the typical structuring of “structured hybrid models”. The functional relationship between the input variables and the output variables is represented by the relationship graph in FIG. 3. The black rectangles represent quantitatively unknown functions here, whereas the white rectangles represent quantitatively known relationships. In order to be able to use the advantages of structured hybrid modeling, it is not necessary for the model to contain known relationships (white rectangles) at all. This knowledge is exploited by the invention for the automatic locating of an SER from descriptors and an effect profile which is determined with respect to a target.

FIG. 4 shows a further preferred exemplary embodiment of the invention in which the individual effect entities can each assume only two states, that is to say logic “zero” and logic “one”, corresponding to “active” or “inactive”.

FIG. 5 shows a flowchart of an embodiment of the method according to the invention. The descriptors of the substances of a substance library for which an effect profile has been determined are provided in step 50. The provision takes place in the form of a file comprising the binary descriptors of the corresponding molecular structures with a uniform length n.

The assignment to the group of the active or inactive molecules has been determined in advance for each of the molecular structures by reference to the effect to be examined; these assignments are provided in the form of the effect profile. The binary descriptors which are provided in step 50 are diversified in step 51, that is to say assigned to the respective effect. Diversification means here that for each possible binary string of descriptors of the lengths it is necessary to know the associated effect.

If this is not the case, with the given data, the diversification must be carried out artificially in a data preprocessing step, either by clustering the data records into individual clusters with a relatively small degree of variation in the molecular structures or by interpolation using a neural network. The clustering enables all the molecular structures in each cluster to be described by means of binary strings with a relatively short length m<n. Within the individual clusters, it is easier to achieve diversification than for the overall conglomeration. An additional possible way of achieving diversification is systematic elimination of correlated substrings from the binary descriptors.

After the diversification in step 51, binary, ternary and univariate variations are calculated in step 52, 53 and 54. This allows a complete system of nonvariant groups in the data set to be calculated. Here, all the tuples composed of variables Vᵢ, Vⱼ of the binary descriptor strings are formed. For each tuple Vᵢ, Vⱼ, two variables are calculated:
the binary variation v2(i,j). It is calculated by
   a) searching for the effect of the overall system for all of the respective combinations of the other parameters for all 4 allocations of the variables (i,j) ((0,0),(0,1),(1,0),(1,1)).
   b) The correlations cor(k,l), k,l=1 . . . 4 of the effect structure between the allocations of (i,j) are then calculated in such a way that an allocation (for example (0,0)) is correlated with another allocation (for example (0,1)) if the effects of the overall system are always identical for both allocations under all variations in the remaining variables. In data records containing errors, the precise identity is not requested but rather a predefined probability that the effects in the variations of the remaining variables are identical. Cor(k,l) is then set to be precisely equal to 1 if the allocation k is correlated, as described, with the allocation 1, and otherwise cor(k,l) is set to 0.
   c) In the next step, the allocations are clustered using known methods in such a way that each cluster contains only allocations which are correlated with one another.
   d) The binary variation v2(i,j) is the number of clusters determined.
the ternary variation v3(i,j;k) which is calculated according to the following algorithm:
   a) The effects for all the respective variations of the remaining variables are sought, for each of the 4 allocations of the variable tuple (i,j) (i,j=1, . . . ,n), and each of the two allocations of the additional variable k.
   b) For each tuple (i,j) and all the variations of the remaining variables, it is checked how the effect changes when there is a jump in the allocation of the variable k from 0 to 1. In the cases in which the effect depends on the allocation of the variable (i,j), it is checked whether the same grouping of the effect in terms of the allocations of (i,j) is present for k=0 and k=1.
   c) The ternary variation v3(i,j;k) is the number of all variations of the remaining variables in which the effect depends on the allocation of the variable (i,j) both for the case in which k=0 and k=1, and in each case different groupings occur in the (i,j) allocations with respect to the effect for k=0 and k=1.
in addition, the variation v1(k) which indicates the number of variations of the remaining variable in which the effect changes if a variable k is changed from 0 to 1 is calculated.

FIG. 6 shows how the procedure is continued from steps 52, 53 and 54.

The functional structure of the SER can be identified unambiguously using the binary and ternary variations v2(i,j) and v3(i,j;k). For this purpose, the irrelevant variables are firstly identified (step 55). Those variables which do not exhibit any influence on the effect whatsoever are referred to as irrelevant variables. These can be identified immediately using v1(k):
a variable k is considered to be irrelevant if v1(k)=0.
All irrelevant variables are eliminated from the input string. Then (step 56), those variable tuples which already form, as tuples, a 2-variable effect entity (2-EE) are identified:
A variable tuple (i,j) which does not contain any irrelevant component forms a 2-EE if
v2(i,j)=2.
Then, it is checked, for all the variables which are not already included in a 2-EE, whether they are included in a more complex effect entity (step 57).
For this purpose, the procedure is continued in accordance with the following algorithm:
a) For all (i,j), the set Mk(i,j) of those k variables for which v3(i,j;k)=0 applies is sought using the associated ternary variations v3(i,j;k), k=1, . . . ,n.
b) All the clusters composed of (i,j) tuples for which each associated cluster element has the same Mk(i,j) set are then sought.
c) All the variables which occur in tuples which belong to the same cluster form an effect entity.

This algorithm allows both the irrelevant variables to be identified from measured data and the functional structure of the SER to be determined in a direct way.

In the case of data which contains noise, i.e. in which the effect assignment to a molecular structure may be faulty, the following modification of the algorithm achieves the goal: In step 55, it is no longer checked whether v1=0, v2=2 and v3=0, but rather a fault bandwidth is permitted. That is to say a variable is deemed to be irrelevant if v1 is less than a predefined limit v1_crit. The compensation of faults in the identification of 2-EEs has already been shown in the description of the identification algorithm. In the identification of complex effect entities, the fault compensation is carried out in such a way that in step a), all the k-variables in Mk(i,j) for which v3(i,j;k) is less than a predefined value v3_crit are set.

This algorithm is a direct method in which the functional structure of the SER is constructed directly from the data. In contrast to indirect methods in which possible structures are tested for compatibility with the data, it has the advantage that the optimum selection of the critical parameters v1_crit, v2_crit and v3_crit is supported by virtue of the fact that the result must be consistent. This means that:
All the variables have to be assigned precisely to one effect entity or defined as an irrelevant variable.
There must not be any overlaps in the assignment.

All the tests have previously shown that when the variable which led to a consistent structure was selected, the correct structure was always generated. The checking of consistency is therefore a powerful test for checking the validity of the functional structure of the SER which is found.

In step 58 of the flowchart in FIG. 6, the consistency of the identified effect entities is checked. If they are not consistent, the selection of correction parameters for the measuring error compensation in step 59 is adapted. The steps 55 and/or 56 and/or 57 are then carried out again and the corresponding results are subjected again to a consistency check in step 58. If they are consistent, the identification of the effect entities is thus terminated.

A preferred exemplary embodiment of the method according to the invention will be explained in more detail below with reference to FIGS. 7 to 11.

FIG. 7 firstly illustrates the procedure for obtaining the experimental data required to carry out this method. The method in FIG. 7 may be carried out largely fully automatically by an automatic laboratory machine.

In step 70, firstly the index p is initialized, that is to say p=0.

In step 71, the descriptor database (cf. database 1 in FIG. 1) is accessed in order to read out the descriptor for substance Sp from the substance library. Overall, a set of q descriptors is present in the database.

In step 72, it is then checked experimentally whether the corresponding substance Sₚ reacts with a target molecule, that is to say exhibits a specific effect or not. If the reaction occurs, the data field Rₚ for the descriptor of the substance Sₚ is set to 1 in step 73, and otherwise the data field Rp is set to 0 in step 74.

Then, in step 75 the value of the index p is incremented. The steps 71, 72 and 73 or 74 are then carried out again for the incremented index, that is to say for the next substance.

The experimentally determined results, that is to say the effect profile, are compiled in a table 80 in FIG. 8. The table 80 contains a descriptor with the variables V₁, V₂, V₃, . . . , Vₙ for each of the substances S₁, S₂, . . . , Sₚ, In addition, each of these descriptors is assigned a data field Rp which specifies, in binary coded form, whether or not a reaction has taken place in the experiment. The data field R₁ which either has the value zero or one is correspondingly assigned to the descriptor for the substance S₁ in the first row of the table 80 depending on whether the substance S₁ has reacted with the target in the experiment or not. The table 80 therefore contains the diversified data (cf. step 51 in FIG. 5).

FIG. 9 shows a flowchart of an embodiment of a method for calculating the binary variations (cf. step 52 in FIG. 9).

In step 90, firstly all the possible two-tuples of variables Vᵢ and Vⱼ where i≠j are formed. If binary descriptors are used which each have a number of n variables V₁, V₂, V₃, . . . , Vₙ, all possible pairings of different variables Vᵢ and Vⱼ are therefore determined.

In step 91, a table is then formed for each of the two-tuples which are determined in step 90. The structure of this table is illustrated in FIG. 10:

FIG. 10 shows a table 100 in which the possible allocations of the variables Vᵢ and Vⱼ serve as the column index. Assuming the use of binary descriptors, for the two variables Vᵢ, Vⱼ there are therefore four different allocation pairs, namely (0,0), (0,1), (1,0), (1,1). The example of such a table 100 shown in FIG. 10 relates here to a two-tuple of variables Vᵢ, Vⱼ where i=1 and j=2.

The possible allocations of remaining variables serve as the row index in table 100. All the variables having an index which is unequal to i and which is unequal to j are referred to as remaining variables here. In the exemplary case under consideration in FIG. 10, these are therefore the remaining variables V₃, V₄, . . . , Vₙ. A specific allocation of these remaining variables is therefore assigned to each row in table 100.

The content of a cell of a specific row and column of table 100 is then obtained as follows:

For the allocation of the remaining variables of the respective row and for the allocation of the two-tuple Vᵢ, Vⱼ of the respective column, table 80 (cf. FIG. 8) is accessed in order to determine the value of the data field Rₚ for this allocation of the variables V₁, V₂, . . . , Vₙ. This value of the data field m Rₚ is then transferred into the respective cell in table 100.

After a table corresponding to table 100 in FIG. 10 has been formed for each of the two-tuples Vᵢ, Vⱼ in step 91 in FIG. 9, the number of different columns is determined for each of these tables in step 92.

In step 93, it is then checked for each of the tables whether the number of different columns of a table under consideration is 1, that is to say it is checked whether the table which is assigned to a specific two-tuple Vᵢ, Vⱼ of variables is composed only of identical columns. If this is the case, it becomes apparent in step 94 that the respective variables Vᵢ, Vⱼ are not relevant.

Otherwise, it is checked for the table under consideration whether the number of different columns is two. If this is the case, it becomes apparent in step 96 that the respective variables Vᵢ and Vⱼ belong to an active entity with precisely two inputs.

Otherwise, in step 97 the ternary variations are formed. The steps 93 and, if appropriate, 95 are carried out for all the tables formed in step 91 in order, as far as possible, to eliminate even at this point variables as irrelevant or to assign variables to an active entity with precisely two inputs. For the variables which are already eliminated as irrelevant in this way, or variables which are assigned to an active entity with precisely two inputs, it is then unnecessary to determine the ternary variations of the step 97. In step 97, all that is therefore necessary is to determine the ternary variations for those variables which could neither be eliminated in step 94 as irrelevant, nor be assigned in step 96 to an active entity with precisely two inputs.

FIG. 1I shows an embodiment for determining the ternary variations (cf. step 97 in FIG. 9).

In step 110, a table in the form of table 100 (cf. FIG. 10) is formed for each two-tuple Vᵢ, Vⱼ, specifically for an allocation of the variable Vₖ to “zero”. Such a table is therefore formed for all three-tuples Vᵢ, Vⱼ and Vₖ, Vₖ always being allocated to zero.

Corresponding tables for each tuple Vᵢ, Vⱼ are formed in step 111, specifically with an allocation of Vₖ=one.

In step 112, it is checked whether for a specific tuple Vᵢ, Vⱼ, that is to say for a specific selection of i and j, the two corresponding tables, that is to say the tables for Vₖ=0 (step 110) and for Vₖ=1 (step 111), are identical. If this is the case, it follows from this in step 113 that the variable Vₖ can be eliminated as irrelevant.

If the opposite is true, in each case the column relation is determined for the two tables under consideration in step 114. The procedure for determining a column relation is to establish, with respect to a particular column in a table, what the relationship is between the elements of this column and corresponding elements of the same row in a different column of the same table, that is to say whether these element pairs are in a relationship of identity or non-identity. These relationships of identity or non-identity are determined for each of the tables in step 114 with respect to all the columns in the respective table.

In step 115, it is then checked whether these column relations in the table pairs for Vₖ=0 and Vₖ=1 which belong to the same two-tuple Vᵢ, Vⱼ of variables are the same. If this is not the case, no definitive conclusion is possible in step 116. If this is the case, it follows from this in step 117 that the variables Vᵢ and Vⱼ are a variable pair candidate for the assignment to the same active entity, it being possible for the active entity to be an active entity with two or more variables. It also follows from this in step 117 that, if the variables Vᵢ, Vⱼ are an applicable variable pair candidate, the variable Vₖ must belong to a different active entity than the active entity of the variables Vᵢ and Vⱼ.

The method in FIG. 11 results in a list of variable pair candidates Vᵢ and Vⱼ as well as in a set of variables Vₖ for each variable pair candidate, which variables Vₖ have to be assigned to another active entity if the respective variable pair candidate is applicable. In the union set of the sets of variables Vₖ which are each assigned to a specific variable pair candidate, contradiction-free clusters of identical sets of variables are then sought. This then results directly in the structure of the pharmacophore which is being sought.

FIG. 12 shows a corresponding result which has been acquired by applying the method in FIG. 11 to a specific application. In the specific application, 360 relevant ternary variations were extracted from 1024 data records. Each descriptor of the data record has a number of ten different variables (V₁, V₂, . . . , V₁₀), and the variable V₂ was identified as irrelevant. The variables V₉ and V₁₀ were identified as belonging to one active entity with precisely two variables (cf. step 96 in FIG. 9).

After elimination of the irrelevant variables and the variables of the two-active entity, the variable pairs Vᵢ and Vⱼ are then the remaining relevant variables tuples left as candidates. These are shown in the upper table in FIG. 12.

In the lower table in FIG. 12, a set of variables Vₖ which belongs to the corresponding row on the upper table of FIG. 2, that is to say to a specific variable pair candidate Vᵢ, Vⱼ, is given in each row. In the lower table in FIG. 12, zero always indicates an empty place. The distribution of the remaining variables was identified from the lower table Mk(i,j) as
effect entity 2: 1 3 4 5
effect entity 3: 6 7 8.

The corresponding cluster is marked in the tables in FIG. 12 by an “x”. The pharmacophore which corresponds to the cluster and has the active entities 4, 5, 6 and 7 is illustrated in FIG. 13. The allocation of the active entity to the variables V₁, V₃, V₄ and V₅ is apparent from the upper table in FIG. 12, and the allocation of the active entity 5 results from the cluster which is formed for the set Mk(i,j). The variables V₉ and V₁₀ are assigned to the active entity with precisely two inputs, and the variable V₂ is not assigned to any active entity as it does not influence the overall effect, that is to say the output of the active entity 7.

### List of reference numerals

| | |
|---|---|
| Database | 1 |
| Database | 2 |
| Pharmacophore | 3 |
| Effect entity | 4 |
| Effect entity | 5 |
| Effect entity | 6 |
| Effect entity | 7 |
| Table | 80 |
| Table | 100 |

1. A method for identifying a pharmacophore having the following steps:
(a)—inputting of descriptors of chemical compounds, each descriptor having a number of variables (V₁, V₂, . . . , Vₙ), and inputting of effects (Rp) assigned to the descriptors,
(b) determining binary variations for two-tuples of variables,
(c) assigning a variable pair (Vᵢ, Vⱼ) to an active entity of the pharmacophore, the active entity having precisely two variables if the binary variation of the variable pair is two,
(d) determining ternary variations to three-tuples of variables (Vᵢ, Vⱼ, Vₖ),
(e) determining variable pair candidates from the ternary variations for assignment to a common active entity, the common active entity having two or more variables, and further determining a set of variables for each variable pair candidate which contains such variables, which, when the variable pair candidate is assigned to the common active entity, have to be assigned to an active entity other than the common active entity, and
(f) determining a conflict-free cluster of sets of the variables for identification of the common active entity.

2. The method of claim 1, wherein the descriptors comprise binary descriptors of a substance library.

3. The method of claim 1, wherein the method further comprises a step for performing data compression on the binary descriptors.

4. The method of claim 1, wherein the effects comprise the effects of the chemical compounds which are respectively assigned to the descriptors on a target molecule, and the effects being binary coded.

5. The method as claimed in claim 1, wherein determining the binary variations and assigning a variable pair to an active entity which has precisely two variables comprises the following steps:
(a) forming two-tuples of variables (Vᵢ, Vⱼ),
(b) forming a table of effects for each of the two-tuples, and using permutations of the remaining variables and possible allocations of the two-tuples of variables as a table index,
(c) determining the number of different columns for each table which is assigned to a two-tuple, and
(d) assigning a two-tuple of variables as a pair of variables to the active entity which has precisely two variables if the number of different columns of a corresponding table is two.

6. The method as claimed in claim 5, wherein the variables of a two-tuple for which the number of different columns of the corresponding table is one being eliminated as irrelevant.

7. The method of claim 5, wherein the ternary variations are determined only if there are tables for which the number of different columns is three or more.

8. The method of claim 1, wherein determining the ternary variations and the variable pair candidates for assignment to a common active entity further comprise the following steps:
(a) forming first tables for two-tuples of variables (Vᵢ, Vⱼ) and for a first effect of a further variable (Vₖ),
(b) forming second tables for two-tuples of variables (Vᵢ, Vⱼ), and for a second effect of a further variable (Vₖ),
(c) determining column relations of the first and second tables with different effects of the further variable, and
(d) determination of variable pair candidates and of a set of variables from the corresponding first and second tables which have identical column relations.

9. The method of claim 8, wherein a further variable is eliminated as irrelevant if the first and second tables of this further variable are essentially the same.

10. The method of claim 8, wherein the set of variables of the conflict-free variable pair candidates are identical in a conflict-free cluster.

11. The method of claim 1, wherein tolerances are permitted in order to eliminate irrelevant variables, to form binary variations and/or to form ternary variations.

12. The method of claim 1, further comprising automatic permissibility limits which yield conflict-free solutions being selected on the basis of searching a three-dimensional parameter space.

13. A computer program having programming means for carrying out the method of claim 1.

14. A computer system having means for carrying out the method of claim 1.
